(19) United States
(12) Patent Application Publication
(10) Pub. No.: US 2004/0210201 A1
(43) Pub. Date: Oct. 21, 2004

(54) Device for maintaining catheter lumen patency

(75) Inventor: Robert C. Farnan, Davie, FL (US)

Correspondence Address: COOK, ALEX, MCFARRON, MANZO,, CUMMINGS & MEHLER, LTD., Suite 2850, 200 West Adams St., Chicago, IL, 60606, US

(73) Assignee: Novoste Corporation

(21) Appl. No.: 10/748,395

(22) Filed: Dec. 30, 2003

Related U.S. Application Data
(60) Provisional application No. 60/437,971, filed on Jan. 2, 2003.

(51) Int. Cl. A61M 5/00

(52) U.S. Cl. 604/246; 604/26

(57) ABSTRACT

The present invention is directed to a method and apparatus for maintaining the patency of vascular catheter lumens so as to keep such lumens open and unobstructed when an indwelling catheter is not being used to administer treatment.

The present application is based on provisional application serial No. 60/437,971 filed Jan. 2, 2003, which is incorporated herein by reference.

### FIELD OF THE INVENTION

The present invention relates to apparatus for maintaining the patency of long term vascular access catheter lumens.

### BACKGROUND OF THE INVENTION

During hemodialysis, chemotherapy, and other similar treatments, patients undergo frequent treatments which utilize the vascular system as the conduit of such treatment. In order to perform such treatments, long term vascular access catheters are placed within the patients' vasculature. Preferably, the catheters remain in place in the vasculature so as to maintain vascular access for such periodic treatments, without having to insert a new catheter before administering each treatment.

However, when fluids are not being administered, such long term vascular access catheters, such as hemodialysis or chemotherapy catheters, have an inherent problem with their lumens becoming clogged by clotting blood. Currently, heparin is injected into the lumen post treatment to minimize the blood clotting within the lumen or at the lumen openings. Additionally, while devices have been designed to remove the clots from catheters, such as miniature brushes, none of these devices effectively address the problem of initial clotting.

Accordingly, it is a general object of the present invention to provide a method and apparatus that addresses one or more of the shortcomings in current devices.

### SUMMARY OF THE INVENTION

The present invention is directed to a method and apparatus for maintaining the patency of vascular catheter lumens so as to keep such lumens open and unobstructed when an indwelling catheter is not being used to administer treatment.

Preferably, in a first embodiment, the device of the present invention comprises a fluid chamber for containing a fluid under pressure, a valve member for controlling the flow of fluid into the chamber, and an outlet from the chamber adapted to communicate with the lumen of an indwelling catheter to keep the lumen open by the controlled flow of fluid from the fluid chamber into the lumen when the catheter is not being used for treatment. The fluid may be gas or liquid. The valve may be in the form of a resealable elastomeric member or other resealable or one-way member. Among other features, the device may also include a fluid permeable filter to prevent particulate matter from being injected into the catheter. The device may further include a luer lock for catheter attachment and a through hole communicating with the fluid chamber to regulate the rate which the fluid leaves the device when the device is attached to a catheter to prevent fluid from escaping from the device when not in use.

The elastomeric member may define a self-sealing port to permit the introduction of fluid into the chamber and prevent the uncontrolled release of fluid.

### BRIEF DESCRIPTION OF THE DRAWINGS

FIG. 1 is a longitudinal cross sectional view of a first embodiment of the present invention.

FIG. 2 is a longitudinal cross sectional view of a second embodiment of the present invention.

### DETAILED DESCRIPTION OF THE PRESENTLY PREFERRED EMBODIMENTS

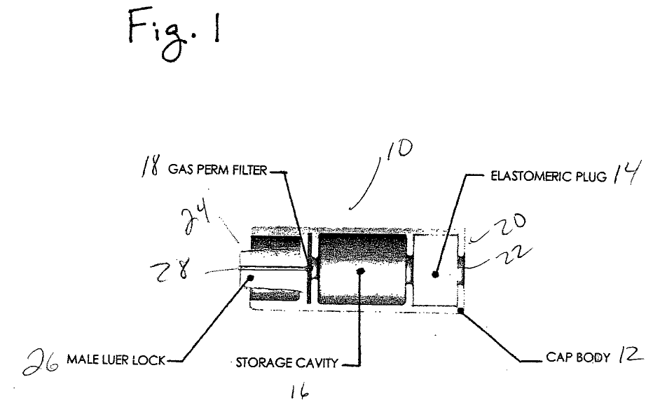

FIG. 1 illustrates a first embodiment of the present invention. The device shown in FIG. 1 is in the form of an integral cap or closure 10 adapted for ready attachment to a catheter hub after the treatment system or apparatus is disconnected and while the catheter remains inserted in the patient's vessel. The cap 10 comprises an outer cap body or housing 12, containing a valve member which may be in the form of an elastomeric member or plug 14, and a fluid chamber, storage cavity or space 16. A fluid permeable filter 18 may also be included in the cap.

Preferably, the cap body 12 defines three regions or cavities. The first region contains or includes the elastomeric member 14 and is located near the proximal or first end 20 of the cap body 12. The second region or cavity is for the fluid chamber 16 and is located between the first and third regions or cavities. This chamber may be defined by the body itself or by a separate member inserted into the body. If the fluid is incompressible liquid, the walls of the chamber may need to be elastomeric to allow for expansion of the chamber when the liquid is introduced and to exert pressure on the liquid The third region or cavity contains the fluid permeable filter 18 and is located in proximity to the distal or second end 24 of the cap body 12. Although described as regions or cavities for purposes of understanding, it is not necessary that there be actual physically separate cavities or regions within the cap. The proximal or first end 20 of the cap body 12 may include a port or opening 22 that allows for the introduction of fluid or gas into the fluid chamber 16.

The distal or second end 24 of the cap body 12 connects to the catheter hub for communication with the catheter lumen and preferably includes a male luer lock 26 with a through hole or passage 28. The through hole 28 is sized to throttle or regulate the fluid exiting the fluid chamber so that the fluid exits the cap at its distal end and enters the catheter lumen at such a rate so as to maintain a low volume “drop” of fluid through the catheter lumen. The low volume “drop” of fluid through the catheter lumen will keep the lumen open when the catheter is not being used for administering treatment. Preferably, the volume of the fluid chamber and rate of fluid exiting the cap and entering the lumen are such that the catheter lumen will remain open for a minimum of three days when treatment is not being administered.

When the cap is not being used and is not connected to a catheter hub, a female luer lock connector (not shown) can be secured to block the passage and prevent the escape of pressurized fluid located in the chamber. The cap body is preferably injection molded rigid plastic or made from other materials of sufficient mechanical strength to allow for secure catheter connection without cracking and sufficient burst strength to handle pressurization, while also permitting sterilization.

Preferably, elastomeric member 14 is formed of an elastomeric material such as latex, neoprene or other similar material. The member 14 seals the proximal port 22 in the cap 10. Member 14 may have a resealable slot or other opening so as to permit a needle or needle-less cannula to be inserted into port 22 and through member 14 to allow for charging or injecting fluid into the fluid chamber 16. If a needle is used to introduce the fluid, it may be inserted through the member 14 without need for a preformed opening, and the material of member 14 will reseal itself, similar to a vial septum. In such case, once the fluid chamber 16 is charged with fluid from the needle, the needle is removed, and the member's elastomeric material seals itself, preventing fluid from escaping from the proximal end of the cap.

Preferably, the fluid chamber 16 within the cap 10 is prefilled with a gas, and more preferably, an inert gas, such as carbon dioxide (C0₂) or N₂, that has characteristics that allow it to be readily absorbed in the patient's body. The fill pressure and volume are such so as to maintain positive pressure (above typical high blood pressure levels) to force fluids from the lumen and to maintain an opening within the catheter lumen when it is not being used to administer treatment. For example, the fill pressure and volume in the fluid chamber 16 should be sufficient so as to force clotting blood and other fluids from the lumen. This will prevent such fluid from remaining in the lumen and allowing clots to form and clog the lumen.

The fluid permeable filter 18 may be of any suitable design and ensures that no particulate matter is injected into the catheter. For example, the filter may be a depth type filter or a membrane filter and may be made of any suitable sterilizable and biocompatible material.

The cap 10 is preferably relatively small and integral and sized similar to currently available injection caps for convenient and unobtrusive attachment to the hub of a catheter. In a preferred embodiment, the caps are disposable and would be replaced after each treatment.

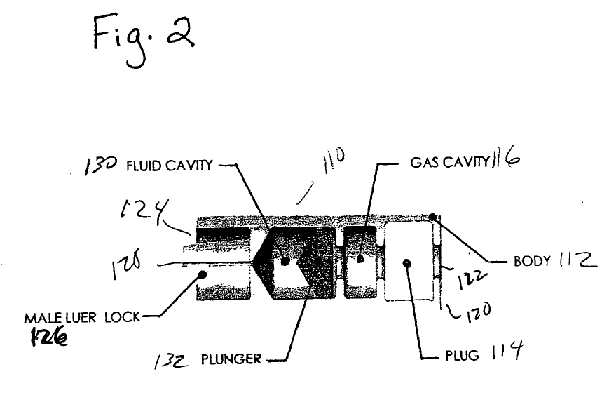

In a second embodiment of the present invention, as illustrated in FIG. 2, the pressurized fluid device or cap 110 preferably comprises a cap housing or body 112, an elastomeric member or plug 114, a gas storage chamber 116, a liquid storage chamber 130, and a fluid tight plunger 132.

In this embodiment, gas, such as that described in the first embodiment, is preferably added to the gas storage chamber 116 in a similar filling and charging process. Biocompatible liquid is added to the liquid storage chamber 130 preferably as another filling and charging process. The liquid selected for filling the liquid storage chamber 130 must be a biocompatible fluid, such as for example heparin or other anti coagulant, saline, sterile water, distilled water, or a mixture of two or more of the above.

The cap body 112 is structured to provide three regions. The first region is for the elastomeric member 114 and is located near the proximal or first end 120 of the cap body 112. The second region is for the gas storage chamber 116 and the inert gas located therein, and is located between the first and third regions. The third region is for the liquid storage chamber 130 and may include both the plunger 132 and the biocompatible liquid therein. The third region is located near the distal or second end 124 of the cap body 112. The plunger 132 and liquid are preferably loaded into the liquid chamber 130 from the distal end 124, which is capped prior to charging the cap with gas. The cap is charged with gas via an injection through the elastomeric member 114 as a finishing operation, in a similar manner to that described above. Preferably, the distal end 124 includes a male luer lock 126 with a through passage 128.

The elastomeric member 114 seals port 122 in the proximal end 120 of the cap in a similar manner to that described supra for the member in the first embodiment.

The plunger 132, which is slidably received within the cap body 112, is in sealed contact with the inside surface and creates a seal between the gas chamber 116 and the liquid chamber 130, which would otherwise be in fluid communication with one another. Further, as the pressurized gas in the gas storage chamber 116 exerts force on the plunger 132, the plunger 132 also serves as a means, much like a syringe piston, for forcing the liquid out of the cap assembly and into the catheter connected to the cap 110. Preferably, the volume of liquid and gas and the size of the fluid passage are such that the fluid is forced out at a rate which will keep the catheter lumen open for a period of time when the catheter is not being used for administering treatment.

The cap body 112, as with the prior embodiment, may include a male luer lock 126 for connecting the cap to a mating catheter luer hub. As noted above, the through hole or passage 128 in the male luer 126 of the cap body 112 is preferably sized so as to regulate delivery of the stored liquid to the catheter lumen for a minimum of three days. When the cap is not being used and is not connected to the catheter hub, a female luer lock member (not shown) can be secured to the male luer lock 126 to prevent the loss of liquid from the cap.

This embodiment, like the first embodiment shown in FIG. 1, also serves to maintain catheter lumen patency. This embodiment of the present invention provides a positive pressure to the catheter lumen as liquid, not gas, is delivered from the cap via the force of the pressurized gas. As a result, a low volume “drop” or flow of biocompatible liquid is provided through the catheter lumen to keep it open when the catheter is not being used for administering treatment.

With either embodiment, in order to prep the catheter for cap acceptance, heparin may still need to be injected into each catheter lumen, as in the current practice. The cap of the present invention would then be attached to each catheter hub thereby providing positive pressure to the lumen and maintaining a fluid flow through the catheter. Preferably, the cap is disposable and replaced after each dialysis or chemotherapy treatment.

It will be understood that the embodiments and examples of the present invention, which have been described, are illustrative of some of the applications of the principles of the present invention. Numerous modifications may be made by those skilled in the art without departing from the true spirit and scope of the invention.

1. A vascular catheter lumen patency device comprising:
a fluid chamber for containing a fluid under pressure;
a valve member for controlling the flow of fluid into the chamber; and
an outlet from the chamber adopted to communicate with the lumen of an indwelling vascular catheter, such that fluid is allowed to exit said chamber at a rate so as to keep open the lumen of an attached catheter when it is not being used for treatment.

2. The device of claim 1 further comprising a fluid permeable filter disposed to prevent particulate matter from being introduced into said catheter lumen.

3. The device of claim 2, wherein said device defines at least three regions, a first region for receiving said valve member, a second region being the fluid chamber, and a third region for receiving said fluid permeable filter.

4. The device of said claim 1 further comprising a self-sealing port in said valve member for allowing introduction of fluid into said fluid chamber and preventing the escape of fluid therefrom.

5. The device of claim 1 wherein said valve member is formed of an elastomeric material.

6. The device of claim 1 further comprising a male luer lock, with a through passageway therein communicating with the fluid chamber for transmitting fluid from the chamber to the lumen of the catheter when connected thereto.

7. The device of claim 6 further comprising a female luer lock connector, wherein when said female luer lock is connected to said male luer lock, fluid is prevented from exiting said fluid chamber.

8. The device of claim 1 wherein said fluid is a gas.

9. The device of claim 8 wherein said gas is an inert gas.

10. The device of claim 9 wherein said gas is selected from the group consisting of CO₂ and N₂.

11. The device of claim 1 wherein the outlet includes a port sized to allow fluid to flow from the fluid chamber at a selected keep open rate.

12. A cap for a vascular catheter lumen comprising:
a cap body with a first end and a second end for connection to said catheter lumen;
an elastomeric plug located at the first end of said cap body;
a fluid storage chamber located adjacent to said elastomeric plug in said cap body; and
a fluid permeable filter and a through hole located at said second end of said cap body and adjacent to said fluid storage chamber,
wherein said fluid storage chamber is filled with a fluid, said elastomeric plug prevents the fluid from exiting said cap through first end, and said fluid is allowed to exit said second end of said cap through said filter and through hole and into said catheter lumen at a rate regulated by said through hole so as to keep said lumen open.

13. The cap of said claim 12 further comprising a port located at the first end of said cap body and located in said plug for filling said fluid into said fluid storage chamber, wherein said elastomeric plug seals itself after introduction of said fluid to prevent said fluid from exiting said first end of said cap.

14. The cap of claim 12 wherein said through hole is located within a male luer lock.

15. The cap of claim 14 further comprising a female luer lock, wherein said female luer lock and said male luer lock combine to prevent fluid from exiting said second end of said cap when said cap is not in use.

16. The cap of claim 12 wherein said fluid is a gas.

17. A device for maintaining patency of a vascular catheter lumen comprising:
a body portion defining a gas chamber;
an elastomeric member in communication with the gas chamber;
a liquid chamber with a liquid tight plunger slidably disposed therein; and
an outlet from said liquid chamber adapted to be placed in communication with a catheter lumen,
wherein the pressure of gas in the gas chamber is applicable against the plunger to force liquid from the liquid chamber through the outlet.

18. The device of claim 17, wherein said device is divided into three regions, a first region for receiving said elastomeric member, a second region being the gas chamber, and a third region defining said liquid chamber and containing said plunger.

19. The device of said claim 17 further comprising a self-sealing port located in the elastomeric member for allowing the introduction of gas into said gas chamber and preventing gas from exiting therefrom.

20. The device of claim 17 further comprising a male luer lock for attachment to a vascular catheter, with a through hole therein sized to regulate the rate in which the liquid leaves said device and enters a catheter lumen.

21. The device of claim 17 wherein said gas is an inert gas.

22. The device of claim 21 wherein said gas is selected from the group consisting of CO₂ and N₂.

23. The device of claim 17 wherein said liquid is biocompatible.

24. The device of claim 23 wherein said liquid is selected from the group consisting of heparin, saline, sterile water, distilled water, and a mixture of two or more of these liquids.
